(19) United States
(12) Patent Application Publication
(10) Pub. No.: US 2005/0282811 A1
(43) Pub. Date: Dec. 22, 2005

(54) Diazabicyclic histamine-3 receptor antagonists

(75) Inventors: Harry R. Howard, Bristol, CT (US); Bishop Wlodecki, Preston, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/152,273

(22) Filed: Jun. 14, 2005

Related U.S. Application Data
(60) Provisional application No. 60/581,845, filed on Jun. 22, 2004.

(51) Int. Cl. A61K 31/498; C07D 487/04

(52) U.S. Cl. 514/249; 544/349

(57) ABSTRACT

This invention is directed to compounds of the formula I and II 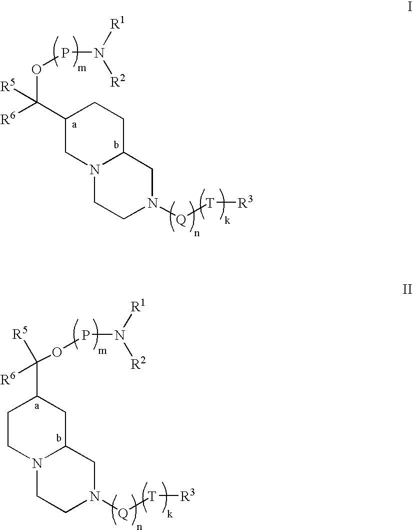 as defined herein, or a pharmaceutically acceptable salt thereof; a pharmaceutical composition containing a compound of formula I or II, a method of treatment of a disorder or condition that may be treated by antagonizing histamine H3 receptors, the method comprising administering to a mammal in need of such treatment a compound of formula I or II as described above, and a method of treatment of a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I or II as described above.

### BACKGROUND OF THE INVENTION

This invention is directed to compounds of formula I and II described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including, for example, generalized anxiety disorder, panic disorder, PTSD, and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders, such as attention-deficit disorders, or other cognitive disorders due to general medical conditions; attention-deficit hyperactivity disorder; psychotic disorders including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the GI tract, hyper- and hypo-motility and acidic secretion of the gastrointestinal tract, sleeping disorders (e.g., hypersomnia, somnolence, and narcolepsy), attention deficit hyperactivity disorder ADHD), hypo- and hyper-activity of the central nervous system (for example, agitation and depression), and other CNS disorders (such as schizophrenia and migraine).

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circ. Res., (1996) 78, 475-481); (Imamura et. al., Circ. Res., (1996) 78, 863-869); (Lin et al., Brain Res. (1990) 523, 325-330); (Monti et al., Neuropsychopharmacology (1996) 15, 31 35); (Sakai, et al., Life Sci. (1991) 48, 2397-2404); (Mazurkiewiez-Kwilecki and Nsonwah, Can. J. Physiol. Pharmacol. (1989) 67, 75-78); (Panula, P. et al., Neuroscience (1998) 44, 465-481); (Wada et al., Trends in Neuroscience (1991) 14,415); (Monti et al., Eur. J. Pharmacol. (1991) 205, 283); (Mazurkiewicz-Kwilecki and Nsonwah, Can. J. Physiol. Pharmacol. (1989) 67, 75-78); (Haas et al., Behav. Brain Res. (1995) 66, 41-44); (De Almeida and Izquierdo, Arch. Int. Pharmacodyn. (1986) 283, 193-198); (Kamei, et al., Psychopharmacology (1990) 102, 312-318); (Kamei and Sakata, Japan. J. Pharmacol. (1991) 57, 437-482); (Schwartz et al., Psychopharmacology; The Fourth Generation of Progress, Bloom and Kupfer (eds.), Raven Press, New York, (1995) 3 97); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234,129); (Yokoyama and linuma, CNS Drugs (1996) 5, 321); (Onodera et al., Prog. Neurobiol. (1994) 42, 685); (Leurs and Timmerman, Prog. Drug Res. (1992) 39,127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4(5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-S51, (1998)); (R. Leurs, R. C. Vollinga and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5(5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17(C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277(2-3):243-50, (1995)); and (Dimitriadou, et al., “Functional relationship between mast cells and C-sensitive nerve fibres evidenced by histamine H3-receptor modulation in rat lung and spleen”, Clinical Science 87(2):151-63, (1994). Such diseases or conditions include cardiovascular disorders such as acute myocardial infarction; memory processes, dementia and cognition disorders such as Alzheimer's disease and attention deficit hyperactivity disorder; neurological disorders such as Parkinson's disease, schizophrenia, depression, epilepsy, and seizures or convulsions; cancer such as cutaneous carcinoma, medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, and WO 02/12224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer's disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO 96/38142. Non-imidazole neuroactive compounds such as beta histamines (Arrang, Eur. J. Pharm. 1985, 111:72-84) demonstrated some histamine H3 receptor activity but with poor potency. EP 978512 and EP 982300 disclose non-imidazole alkylamines as histamine H3 receptor antagonists. WO 02/12224 (Ortho McNeil Pharmaceuticals) describes non-imidazole bicyclic derivatives as histamine H3 receptor ligands, and EP 1275647 (Les Laboratoires Servier) has disclosed novel octahydro-2H-pyrido[1,2-a]pyrazines that are selective H3 receptor antagonists. Other receptor antagonists have been described in WO 02/32893 and WO 02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of diazabicyclic compounds exhibits a high and specific affinity to the histamine H3 receptor.

### SUMMARY OF THE INVENTION

This invention is directed to compounds of the formula I and II: 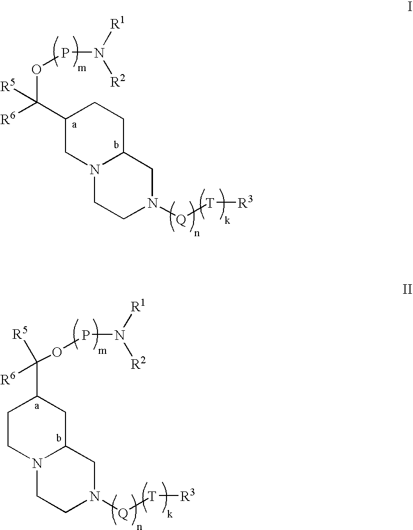
or the pharmaceutically acceptable salt(s) thereof, wherein:

P is independently methylene (optionally substituted at available positions on the carbon atom by hydrogen, fluorine, OH, carbonyl, C₁-C₈ alkylene); or a 3-8 membered cycloalkyl ring (e.g., cyclopropyl, cyclobutyl, cyclopentyl, cyclohexyl);

m=2, 3 or 4;

Q is independently selected from C₁-C₆ alkylene and may be substituted at available positions by R^{a} wherein R^{a} is selected from hydrogen, C₁-C₆ alkyl or C₆-C₁₄ aryl;

T is selected from the group consisting of methylene, C═O, —C(═O)—N, SO₂;

n=0, 1, 2, or 3;

k =0, 1;

R¹ and R² are independently selected from the group consisting of hydrogen;

C₁-C₈ alkyl optionally substituted with 1 to 4 halogens (especially fluorine) or OH;

C₃-C₇ cycloalkyl;

C₆-C₁₄ aryl;

3-8 membered heterocycloalkyl optionally substituted with a C₁-C₄ alkyl-carbonyl group;

C₆-C₁₀arylsulfonyl optionally substituted with C₁-C₂ alkyl; and

5-10 membered heteroaryl; or

NR¹ together with an available carbon atom in P form a 4-8 membered cyclic ring, and said ring may include up to two additional heteroatoms selected from N, O or S;

R³ is selected from the group consisting of hydrogen;

phenyl, naphthyl or heteroaryl, each of which may be optionally substituted by one or more of X; wherein

X is H, F, Cl, Br, I, CN, OH, OR⁷, S(O)ₜR⁸, COR⁹, CONR¹⁰R¹¹; or

R¹ and R² together with the nitrogen of the NR¹R² group form a 4-8 member ring, wherein from one to three of the carbons in the ring is optionally replaced by O, S, NR⁴, or CO, and the ring is optionally fused to a C₆-C₁₀ arylene and is optionally substituted at a ring carbon with one or two C₁-C₄ alkyl groups;

R⁴ is hydrogen;

C₁-C₈ alkyl optionally substituted with 1 to 4 halogens;

5-10 membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, and cyano;

C₆-C₁₀ aryl optionally substituted with one or two C₁-C₂ alkyl; or

C₁-C₄ alkyl-carbonyl;

R⁵ and R⁸ are independently selected from the group consisting of hydrogen;

fluorine;

C₁-C₆ alkyl (optionally substituted by F); or

R⁵ and R⁶ together with the carbon atom to which they are attached form a cycloalkyl ring of 3 to 7 atoms;

R⁷, R⁸ and R⁹ are independently selected from the group consisting of C₁-C₈ alkyl optionally substituted with 1 to 4 halogens;

5-10 membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, and cyano;

C₆-C₁₀ aryl optionally substituted with one or two C₁-C₂ alkyl;

R¹⁰ and R¹¹ are independently selected from the group defined as R¹ and R² above; and

t is 0, 1 or 2.

Where cis and trans isomers are possible for an embodiment of the inventive compounds of formula I and II, both cis and trans isomers are within the scope of the invention.

The term “alkyl” refers to straight or branched chains of carbon atoms. Exemplary alkyl groups are C₁-C₆ alkyl groups which include methyl, ethyl, propyl, isopropyl, butyl, isobutyl, pentyl, isopentyl, hexyl, and the like, including all regioisomeric forms thereof, and straight and branched chain forms thereof. The term “alkyl” is also used to denote straight or branched chains of carbon atoms having one or more carbon-carbon double bonds, such as vinyl, allyl, butenyl, and the like, as well as straight or branched chains of carbon atoms having one or more carbon-carbon triple bonds, such as ethynyl, propargyl, butynyl, and the like. The term “aryl” denotes a cyclic, aromatic hydrocarbon. Examples of aryl groups include phenyl, naphthyl, anthracenyl, phenanthrenyl, and the like. The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon-carbon double bonds in the ring. Examples of cycloalkyl groups include cyclopropyl, cyclobutyl, cyclopentyl, cyclohexyl, cycloheptyl, and the like, as well as cyclopentenyl, cyclopentadienyl, cyclohexenyl, cyclohexadienyl, and the like. As used herein, the term “cycloalkyl” is also intended to denote a cyclic group comprising at least two fused rings, such as adamantanyl, decahydronaphthalinyl, norbornanyl, where the cyclic group may also have one or more carbon-carbon double bonds in one or both rings, such as in bicyclo[4.3.0]nona-3,6(1)-dienyl, dicyclopentadienyl, 1,2,3,4-tetrahydronaphthalinyl (tetralinyl), indenyl, and the like. The term “halogen” represents chloro, fluoro, bromo, and iodo. The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- to fourteen-member rings that contain from one to three heteroatoms independently selected from oxygen, nitrogen, and sulfur. Examples of preferred heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, and xanthenyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “C₀-C₄” includes the embodiment where there are no carbons in a chain. Thus, for example, the groups “C₃-C₇ cycloalkyl-C₀-C₄ alkyl,” “C₆-C₁₄ aryl-C₀-C₄ alkyl,” “5-10-membered heteroaryl-C₀-C₄ alkyl,” and “C₆-C₁₄ aryl-C₀-C₄ alkylene-O—C₀-C₄ alkyl” include C₃-C₇ cycloalkyl, C₆-C₁₄ aryl, 5-10-membered heteroaryl, and C₆-C₁₄ aryl-O—C₀-C₄ alkyl, respectively.

The term “C₁-C₄ dialkylamino” refers to a dialkylamino group in which each alkyl group is independently a C₁-C₄ alkyl group.

This invention is also directed to:

a pharmaceutical composition for treating, for example, a disorder or condition that may be treated by antagonizing histamine-3 receptors, the composition comprising a compound of formula I or II as described above, and optionally a pharmaceutically acceptable carrier;

a method of treatment of a disorder or condition that may be treated by antagonizing histamine-3 receptors, the method comprising administering to a mammal in need of such treatment a compound of formula I or II as described above; and

a pharmaceutical composition for treating, for example, a disorder or condition selected from the group consisting of depression, mood disorders, schizophrenia, anxiety disorders, Alzheimer's disease, attention deficit disorder (ADD), attention deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper- and hypo-motility and acidic secretion of the gastrointestinal tract, the composition comprising a compound of formula I or II as described above, and optionally a pharmaceutically acceptable carrier.

This invention is also directed to a method of treatment of a disorder or condition selected from the group consisting of the disorders or conditions listed in the preceding paragraph, the method comprising administering to a mammal in need of such treatment a compound of formula I or II as described above.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I or II as described above.

The disclosed compounds may also be used as part of a combination therapy, including their administration as separate entities or combined in a single delivery system, which employs an effective dose of a histamine H3 antagonist compound of general formula I or II and an effective dose of a histamine H1 antagonist, such as cetirizine (Zyrtec™), for the treatment of allergic rhinitis, nasal congestion and allergic congestion.

The disclosed compounds may also be used as part of a combination therapy, including their administration as separate entities or combined in a single delivery system, which employs an effective dose of a histamine H3 antagonist compound of general formula I or II and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating depression and mood disorders.

The compounds of the present invention may have optical centers and therefore may occur in different enantiomeric configurations. Formulae I and II, as depicted above, include all enantiomers, diastereomers, and other stereoisomers of the compounds depicted in structural formula I and II, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I and 1I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹³F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I and II of this invention and prodrugs thereof can generally be prepared by carrying out the procedures disclosed in the Schemes and/or in the Examples and Preparations below, by substituting a readily available isotopically labeled reagent for a non-isotopically labeled reagent.

“Antagonizing histamine-3 (H3) receptors,” as used herein, refers to acting as a histamine-3 receptor antagonist.

A “unit dosage form” as used herein is any form that contains a unit dose of the compound of formula I or II. A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., attention deficit hyperactivity disorder) in the average human are preferably arranged so that each metered dose or “puff” of aerosol contains 20 μg to 1000 μg of the compound of the invention. The overall daily dose with an aerosol will be within the range 100 μg to 100 mg. Administration may be several times daily, for example 2, 3, 4 or 8 times, giving for example, 1, 2 or 3 doses each time.

In connection with the use of an active compound of this invention with a histamine H1 antagonist, preferably cetirizine, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I and II are present in such dosage forms at concentration levels ranging from about 0.5% to about 95% by weight of the total composition, i.e., in amounts which are sufficient to provide the desired unit dosage and a histamine H1 antagonist, preferably cetirizine, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a histamine H1 antagonist) for oral, parenteral, rectal or buccal administration to the average adult human for the treatment of the conditions referred to above is from about 0.01 mg to about 2000 mg, preferably from about 0.1 mg to about 200 mg of the active ingredient of formula I or II per unit dose which could be administered, for example, 1 to 4 times per day.

A proposed daily dose of a histamine H1 antagonist, preferably cetirizine, in the combination formulation for oral, parenteral or buccal administration to the average adult human for the treatment of the conditions referred to above is from about 0.1 mg to about 2000 mg, preferably from about 1 mg to about 200 mg of the histamine H1 antagonist per unit dose which could be administered, for example, 1 to 4 times per day.

A preferred dose ratio of cetirizine to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a histamine H1 antagonist, preferably cetirizine, preferably from about 1 mg to about 200 mg of cetirizine. Administration may be several times daily, for example 2, 3, 4 or 8 times, giving for example, 1, 2 or 3 doses each time.

As previously indicated, a histamine H1 antagonist, preferably cetirizine, in combination with compounds of formula I or II are readily adapted to therapeutic use as antiallergy agents. In general, these antiallergy compositions containing a histamine Hi antagonist, preferably cetirizine, and a compound of formula I or II are normally administered in dosages ranging from about 0.01 mg to about 100 mg per kg of body weight per day of a histamine H1 antagonist, preferably cetirizine, preferably from about 0.1 mg. to about 10 mg per kg of body weight per day of cetirizine; with from about 0.001 mg. to about 100 mg per kg of body weight per day of a compound of formula I or II, preferably from about 0.01 mg to about 10 mg per kg of body weight per day of a compound of formula I or II, although variations will necessarily occur depending upon the conditions of the subject being treated and the particular route of administration chosen.

In connection with the use of an active compound of this invention with a 5-HT re-uptake inhibitor, preferably sertraline, for the treatment of subjects possessing any of the above conditions, it is to be noted that these compounds may-be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I and II are present in such dosage forms at concentration levels ranging from about 0.5% to about 95% by weight of the total composition, i.e., in amounts which are sufficient to provide the desired unit dosage and a 5-HT re-uptake inhibitor, preferably sertraline, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I or II per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of formula I or II are readily adapted to therapeutic use as antidepressant agents. In general, these antidepressant compositions containing a 5-HT re-uptake inhibitor, preferably sertraline, and a compound of formula I or II are normally administered in dosages ranging from about 0.01 mg to about 100 mg per kg of body weight per day of a 5-HT re-uptake inhibitor, preferably sertraline, preferably from about 0.1 mg. to about 10 mg per kg of body weight per day of sertraline; with from about 0.001 mg. to about 100 mg per kg of body weight per day of a compound of formula I or II, preferably from about 0.01 mg to about 10 mg per kg of body weight per day of a compound of formula I or II, although variations will necessarily occur depending upon the conditions of the subject being treated and the particular route of administration chosen.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A compound of formula I or II, which is basic in nature, is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogen phosphate, naphthalene-1-sulfonate, naphthalene-2-sulfonate, oxalate, phenylbutyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts.

Preferred embodiments of the present invention include the compounds of formula I and II in which

(A) R¹ is methyl, R² is methyl and R³ is phenyl; or

(B) R¹ and R² together with the nitrogen to which they are attached form the 5-membered pyrrolidine ring, and R³ is phenyl; or

(C) R¹ and R² together with the nitrogen to which they are attached form a 5-membered pyrrolidine ring, and the groups at carbon positions a and b are trans; or

(D) R¹ and R² together with the nitrogen to which they are attached form the 6-membered piperidine ring, and R³ is phenyl, and the groups at position a and b are trans.

(E) R¹ and R² together with the nitrogen to which they are attached form the 6-membered piperidine ring, and R³ is phenyl, and the groups at position a and b are cis.

The most preferred embodiments of the present invention include the compounds of formula I and II in which R¹ and R² together with nitrogen to which they are attached form the 6-membered piperidine ring and R³ is phenyl and the groups at position a and b are trans.

Preferred embodiments of the present invention also include any combination of the foregoing embodiments (A)-(E).

Preferred compounds of formula I or II in accordance with the present invention are the following:

2-(4-Methanesulfonylbenzyl)-7-(3-morpholin-4-ylpropoxymethyl)-octahydro-pyrido[1,2-a]pyrazine;

7-[3-(2,5-Dimethylpyrrolidin-1-yl)-propoxymethyl]-2-(4-methanesulfonylbenzyl)-octahydro-pyrido[1,2-a]pyrazine;

2-(4-Methanesulfonylbenzyl)-7-[3-(4-methylpiperazin-1-yl)-propoxymethyl]-octahydro-pyrido[1,2-a]pyrazine;

2-(4-Methanesulfonylbenzyl)-7-[3-(4-phenylpiperazin-1-yl)-propoxymethyl]-octahydro-pyrido[1,2-a]pyrazine;

{3-[2-(4-Methanesulfonylbenzyl)-octahydro-pyrido[1,2-a]pyrazin-7-ylmethoxy]-propyl}-dimethylamine;

7-(3-Azetidin-1-yl-propoxymethyl)-2-(4-methanesulfonylbenzyl)-octahydro-pyrido[1,2-a]pyrazine;

2-(4-Methanesulfonylbenzyl)-7-(3-thiomorpholin-4-ylpropoxymethyl)-octahydro-pyrido[1,2-a]pyrazine;

7-[3-(3,4-Dihydro-1H-isoquinolin-2-yl)-propoxymethyl]-2-(4-methanesulfonylbenzyl)-octahydro-pyrido[1,2-a]pyrazine;

7-[3-(3,4-Dihydro-2H-quinolin-1-yl)-propoxymethyl]-2-(4-methanesulfonylbenzyl)-octahydro-pyrido[1,2-a]pyrazine;

7-[3-(2,6-Dimethyl-piperidin-1-yl)-propoxymethyl]-2-(4-methanesulfonylbenzyl)-octahydro-pyrido[1,2-a]pyrazine;

2-(4-Methanesulfonylbenzyl)-7-(2-piperidin-1-ylethoxymethyl)-octahydro-pyrido[1,2-a]pyrazine;

2-(4-Methanesulfonylbenzyl)-7-(2-morpholin-4-ylethoxymethyl)-octahydro-pyrido[1,2-a]pyrazine;

(4-Methanesulfonylphenyl)-[7-(3-piperidin-1-ylpropoxymethyl)-octahydro-pyrido[1,2-a]pyrazin-2-yl]-methanone;

2-(4-Methanesulfonylbenzenesulfonyl)-7-(3-piperidin-1-ylpropoxymethyl)-octahydro-pyrido[1,2-a]pyrazine;

7-(3-Piperidin-1-ylpropoxymethyl)-octahydro-pyrido[1,2-a]pyrazine-2-carboxylic acid (4-methanesulfonylphenyl)-amide;

Phenyl-[8-(3-piperidin-1-ylpropoxymethyl)-octahydro-pyrido[1,2-a]pyrazin-2-yl]-methanone;

2-Benzenesulfonyl-8-(3-morpholin-4-ylpropoxymethyl)-octahydro-pyrido[1,2-a]pyrazine;

8-(3-Morpholin4-ylpropoxymethyl)-2-(pyridine-4-sulfonyl)-octahydro-pyrido[1,2-a]pyrazine;

(2-Methylpyrimidin-5-yl)-[8-(3-morpholin-4-ylpropoxymethyl)-octahydro-pyrido[1,2-a]pyrazin-2-yl]-methanone;

1-(4-{1-[8-(3-Morpholin-4-ylpropoxymethyl)-octahydro-pyrido[1,2-a]pyrazin-2-yl]-ethyl}-phenyl)-ethanone;

Cyclopropyl-(4-{7-[1-(3-piperidin-1-ylpropoxy)-ethyl]-octahydro-pyrido[1,2-a]pyrazin-2-ylmethyl}-phenyl)-methanone;

7-(3-Piperidin-1-ylpropoxymethyl)-2-quinolin-8-ylmethyl-octahydro-pyrido[1,2-a]pyrazine;

5-[7-(3-Piperidin-1-ylpropoxymethyl)-octahydro-pyrido[1,2-a]pyrazin-2-ylmethyl]-indan-1-one; and

7-[7-(3-Piperidin-1-ylpropoxymethyl)-octahydro-pyrido[1,2-a]pyrazin-2-ylmethyl]-chroman-4-one.

The most preferred examples of compounds according to the present invention include:

(7R, 9aS)-7-(3-Piperidin-1-ylpropoxymethyl)-octahydro-pyrido[1,2-a]pyrazine;

(7R, 9aS)-2-Methyl-7-(3-piperidin-1-ylpropoxymethyl)-octahydro-pyrido[1,2-a]pyrazine;

(7R, 9aS)-2-(3-Phenylpropyl)-7-(3-piperidin-1-ylpropoxymethyl)-octahydro-pyrido[1,2-a]pyrazine;

(7R, 9aS)-2-Phenethyl-7-(3-piperidin-1-ylpropoxymethyl)-octahydro-pyrido[1,2-a]pyrazine;

(7R, 9aS)-2-Benzyl-7-(3-piperidin-1-ylpropoxymethyl)-octahydro-pyrido[1,2-a]pyrazine;

(7R, 9aS)-1-{4-[7-(3-Piperidin-1-ylpropoxymethyl)-octahydro-pyrido-[1,2-a]pyrazin-2-ylmethyl]-phenyl}-ethanone;

(7R, 9aS)-2-(4-Methoxybenzyl)-7-(3-piperidin-1-ylpropoxymethyl)-octahydro-pyrido[1,2-a]pyrazine;

(7R, 9aS)-2-(3-Methoxybenzyl)-7-(3-piperidin-1-ylpropoxymethyl)-octahydro-pyrido[1,2-a]pyrazine;

(7R, 9aS)-2-(4-Fluorobenzyl)-7-(3-piperidin-1-ylpropoxymethyl)-octahydro-pyrido[1,2-a]pyrazine;

(7R, 9aS)-N-{4-[7-(3-Piperidin-1-ylpropoxymethyl)-octahydro-pyrido-[1,2-a]pyrazin-2-ylmethyl]-phenyl}-acetamide;

(7R, 9aS)-2-(4-Chlorobenzyl)-7-(3-piperidin-1-ylpropoxymethyl)-octahydro-pyrido[1,2-a]pyrazine;

(7R, 9aS)-2-(3-Chlorobenzyl)-7-(3-piperidin-1-ylpropoxymethyl)-octahydro-pyrido[1,2-a]pyrazine;

(7R, 9aS)-2-(3-Fluorobenzyl)-7-(3-piperidin-1-ylpropoxymethyl)-octahydro-pyrido[1,2-a]pyrazine;

(7R, 9aS)-2-(4-Methanesulfonylbenzyl)-7-(3-piperidin-1-ylpropoxy-methyl)-octahydro-pyrido[1,2-a]pyrazine;

(7S, 9aS)-1-{4-[7-(3-Piperidin-1-ylpropoxymethyl)-octahydro-pyrido-[1,2-a]pyrazin-2-ylmethyl]-phenyl}-ethanone;

(7S, 9aS)-2-(4-Chlorobenzyl)-7-(3-piperidin-1-ylpropoxymethyl)-octahydro-pyrido[1,2-a]pyrazine;

(7R, 9aS)-7-(3-Piperidin-1-ylpropoxymethyl)-2-pyridin-3-ylmethyl-octahydro-pyrido[1,2-a]pyrazine;

(7R, 9aS)-7-(3-Piperidin-1-ylpropoxymethyl)-2-pyridin-4-ylmethyl-octahydro-pyrido[1,2-a]pyrazine;

(7R, 9aS)-7-(3-Piperidin-1-ylpropoxymethyl)-2-pyridin-2-ylmethyl-octahydro-pyrido[1,2-a]pyrazine;

(7S, 9aS)-2-(4-Methanesulfonylbenzyl)-7-(3-piperidin-1-ylpropoxy-methyl)-octahydro-pyrido[1,2-a]pyrazine;

(8R, 9aS)-2-(4-Methanesulfonylbenzyl)-8-(3-piperidin-1-ylpropoxy-methyl)-octahydro-pyrido[1,2-a]pyrazine; and

(8R, 9aS)-1-{4-[8-(3-Piperidin-1-ylpropoxymethyl)-octahydro-pyrido-[1,2-alpyrazin-2-ylmethyl]-phenyl}-ethanone.

### DETAILED DESCRIPTION OF THE INVENTION

The compounds of formula I according to the invention may be prepared by the general procedure shown in Scheme 1. Details for the syntheses of trans (7R, 9aS) octahydro-pyrido[1,2a]pyrazine analogs are presented in the following descriptions; however, the remaining trans and cis diastereomers, as well as the corresponding isomers of general formula II, can be prepared in a similar manner using the methods and procedures described in this application as well as those found in the chemical literature.

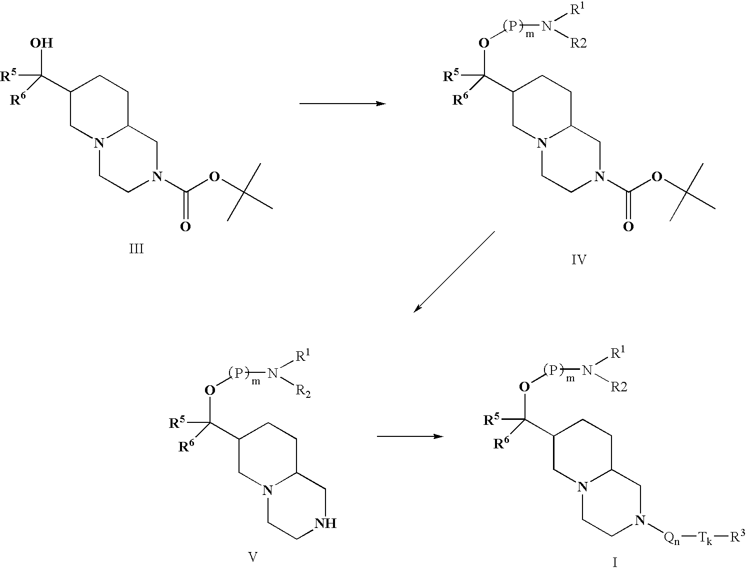

In Scheme 1, compounds of the formula I are prepared as follows. An intermediate of the general formula III, prepared as described by Sanner, et al (Bioorganic & Medicinal Chemistry Letters, 8:725-730 (1998)) is reacted with a compound of the general formula (VII):
L¹-(P)ₘ—NR¹R² (VII)
wherein m, P, R¹ and R² are as previously defined and L¹ is defined as a reactive leaving group such as chlorine, bromine, hydroxyl, mesylate, tosylate and the like to generate an intermediate of the general formula IV. In the instances when the reaction involves an alcohol (i.e., L¹=OH), then conditions associated with the Mitsunobu reaction can effectively be used to prepare the intermediate IV. When the reaction involves a halogen (e.g., Cl, Br, I) or a reactive ester such as a mesylate (i.e., methanesulfonate) or tosylate (i.e., para-toluenesulfonate) derivative, then the conditions associated with ether formation (e.g., an acid scavenger such as sodium or potassium carbonate, sodium or potassium hydroxide, sodium or potassium tert-butoxide, sodium or potassium hydride, n-butyl lithium and the like, and preferably potassium tert-butoxide, in a reaction inert solvent such as diethyl ether, THF, DMF, DMA, preferably in THF) can be effectively utilized to prepare this intermediate. The reactions can be conducted within the temperature range of about −78° C. to about 150° C., and preferably from about 20° C. to about 60° C. at a pressure of about one atmosphere to about three atmospheres of pressure, preferably at atmospheric pressure and preferably under an inert nitrogen or argon gas atmosphere.

Alternatively, the intermediate of formula III may be reacted with a reagent of the formula VII:
L¹-(P)ₘ-L² (VII)
wherein P, m and L¹ are as defined above and L² is a second leaving group chosen from the list which includes chlorine, bromine, iodine, mesylate, tosylate and the like. Conditions for reacting these two compounds are selected such that the intermediate product so obtained (i.e., IX): 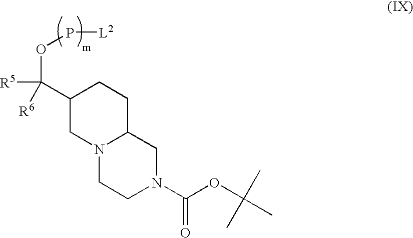
is capable of further reaction with a primary or secondary amine (e.g., H₂NR¹ or HNR¹R², respectively) to generate an intermediate of general formula IV.

The tert-butoxycarbonyl group, which serves as a protecting group during the formation of intermediate IV is then removed under acidic conditions to produce an intermediate amine of general formula V. Such reaction conditions to remove this group are described in Protective Groups in Organic Synthesis (T. W. Greene and P. G. M. Wuts, John Wiley and Sons, Inc., NY, 1991, 2nd Ed., pp. 327-330). These include the use of acidic reaction conditions including 3M HCl in ethyl acetate or dioxane at room temperature, trifluoroacetic acid (TFA) with or without a solvent at room temperature, trimethylsilyl iodide (TMSI) or chloride (TMSCl) in CHCl₃ or CH₃CN at room temperature, and 10% aqueous H₂SO₄ in dioxane. It should also be recognized that the tert-butoxycarbonyl group present in the intermediates of formulae III and IV is not the only protecting group that can be effectively used in this process, as there are others (e.g., as described in the Greene and Wuts reference above) that also may be suitable and preferable depending on the nature and stability of the groups attached to the intermediates of formulae III and IV.

After removal of the protecting group from intermediate IV, the intermediate of formula V may then be converted to the novel compounds of formula I. This transformation can be made using a variety of reagents and methods familiar to those skilled in the art of organic synthesis or readily prepared using techniques disclosed in, or adapted from, the chemical and patent literature. For example, an intermediate of formula V may be reacted with an alkylating reagent of the general formula X:
L³-(Q)ₙ-(T)ₖ-R³ (X)
wherein the groups Q, T, n, k and R³ are as defined above and L³ is a leaving group such as chlorine, bromine, iodine, mesylate, tosylate and the like. These can be reacted under conditions similar to those described above for the conversion of intermediates of formula III to IV.

Alternatively, the intermediate V can be reacted under reductive amination conditions with an aldehylde or ketone of the general formula XI:
R^{a}—C(═O)-(Q)ₙ₋₁-Tₖ-R³ (XI)
wherein k, n, Q, T, R³ and R^{a} are as defined previously. This method may be performed in a single, concerted process (e.g., see A. F. Abdel-Magid, C. A. Maryanoff and K. G. Carson in Tetrahedron Letters, 1990, 39:5595-5598). In such conversions, the carbonyl compound of formula XI and the intermediate amine of formula V are combined in a reaction inert solvent and treated with reagents like sodium cyanoborohydride or sodium triacetoxyborohydride. Suitable solvents include, among others, tetrahydrofuran (THF) and 1,2-dichloroethane (DCE) and the reactions may be conducted with or without the addition of an organic acid (e.g., acetic acid). The conversion of compounds of formula V to compounds of formula I can also be completed using two or more individual steps, involving the initial formation of an imine intermediate such as XII, followed by reduction of the C═N double bond to generate the compounds of general formula I.

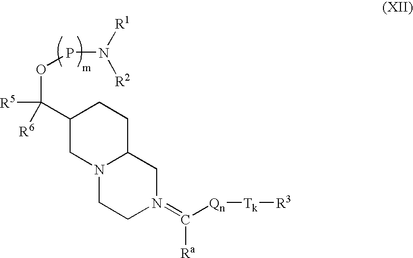

For example, the intermediate of formula V and the appropriate aldehyde (R^{a}═H) or ketone of formula XI can be combined in the presence of a dehydrating reagent in a reaction neutral solvent like benzene, toluene, methanol or ethanol and stirred for a prescribed amount of time until the reaction is judged to be completed (e.g., using techniques like thin layer chromatography (tlc), mass spectrometry (MS) or nuclear magnetic resonance spectrometry (NMR)). Such dehydrating reagents include, for example, p-toluenesulfonic acid, titanium(IV) chloride, titanium(IV) isopropoxide or molecular sieves. The reaction can be conducted within the range of about 0° C. to about the boiling point of the solvent employed and at pressures of about one to about three atmospheres. The intermediate imine XII so obtained can then be reduced with a variety of reagents and under a variety of conditions familiar to one skilled in the art, including the use of hydrogen gas in the presence of a catalyst like palladium on carbon (Pd/C) or platinum on carbon (Pt/C), as well as with sodium borohydride, sodium (triacetoxy)borohydride, sodium cyanoborohydride and the like. The use of hydrogen as the reducing agent is often conducted in a reaction inert solvent such as methanol, ethanol, THF, 1,4-dioxane and similar solvents at a pressure of about one atmosphere to a pressure of about 5 atmospheres of hydrogen and typically at a temperature from about room temperature to a temperature that is below the boiling point of the solvent employed. When using the hydride reagents, the choice of solvent can be made from, but not limited to, methanol, ethanol, isopropanol, 1,4-dioxane, THF and the like. The reaction can be carried out at atmospheric pressure and at temperatures ranging from about −40° C. to about the boiling temperature of the solvent employed, typically at 0-40° C. and most preferably at room temperature.

For the preparation of the carboxamide and sulfonamide compounds of formula I (i.e., wherein T is —C(═O)— or —S(═O)₂— and n=0, respectively), a compound of formula V may be reacted with an appropriate R³-carbonyl chloride or R³-sulfonyl chloride, respectively, in a reaction inert solvent (e.g., chloroform, dichlormethane, etc.) in the presence of an acid scavenger such as sodium or potassium carbonate.

For the preparation of the corresponding urea compounds of formula I (i.e., wherein T is —C(═O)—NH—) a compound of formula V may be reacted with an appropriate isocyanate (i.e., R³—NCO) in a reaction inert solvent (e.g., chloroform, dichlormethane, THF and the like).

In the examples that follow, the abbreviations used are intended to have the following, general meaning:

bm: broad multiplet (NMR)

bs: broad singlet (NMR)

dd: doublet of doublets (NMR)

d.e.: diatomaceous earth, filter agent

DMF: dimethyformamide

LRMS: low resolution mass spectrometry

calcd; calculated

d; doublet (NMR)

EtOAc: ethyl acetate

J: coupling constant (NMR)

LAH: lithium aluminum hydride

m: multiplet (in NMR)

min: minute(s)

m/z: mass to charge ratio (in mass spectrometry)

obsd: observed

Rf: retention factor (in chromatography)

Rt: retention time (in chromatography)

rt: room temperature

s: singlet (NMR), second(s)

t: triplet

THF: tetrahydrofuran

tlc: thin layer chromatography

Solvents were purchased and used without purification. Yields were calculated for material judged homogenous by thin layer chromatography and NMR. Thin layer chromatography was performed on Merck Kieselgel 60 F 254 plates eluting with the solvents indicated, visualized by a 254 nm UV lamp, and stained with either an aqueous KMnO₄ solution or an ethanolic solution of 12-molybdophosphoric acid. Flash column chromatography was performed with using either pre-packed Biotage® or ISCO® columns using the size indicated. Nuclear magnetic resonance (NMR) spectra were acquired on a Unity 400 or 500 at 400 MHz or 500 MHz for ¹H, respectively, and 100 MHz or 125 MHz for ¹³C NMR, respectively. Chemical shifts for proton ¹H NMR spectra are reported in parts per million relative to the singlet of CDCl₃ at 7.24 ppm. Chemical shifts for ¹³C NMR spectra are reported in parts per million downfield relative to the centerline of the triplet of CDCl₃ at 77.0 ppm. Mass spectra analyses were performed on a APCI Gilson 215, micromass ZMD (50% Acetonitrile/50% water) spectrometer.

Reactions under microwave conditions were done using 2-5 mL round bottom vials, fitted with septa. The vials containing the reactants were inserted into the reaction chamber of a EMRYS™ Creator microwave apparatus (maximum power of 300 W) from Personal Chemistry Inc., 25 Birch St., Bldg C, Suite 304, Milford, Mass. 01757 and heated to the appropriate temperature for a the prescribed period of time. HPLC was performed according to the following methods:

Method A: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 100% solvent A, 0% solvent B, flow 20; time 2.0, 100% solvent A, 0% solvent B, flow 20; time 12.0, 0% solvent A, 100% solvent B, flow 20; time 15.0, 0% solvent A, 100% solvent B, flow 20; time 15.1, 100% solvent A, 0% solvent B, flow 20; time 20.0, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method B: Preparative conditions (Waters 600 & Waters 2767 Sample Manager); Column: Waters Xterra PrepMS C₁₈ column, 5 μm, 30×150 mm steel column, part # 186001120, serial # T22881T 09; solvent A—0.1% Trifluoroacetic acid/water; solvent B—Acetonitrile; volume of injection: 1050 μL; time 0.0, 100% solvent A, 0% solvent B, flow 20; time 2.0, 100% solvent A, 0% solvent B, flow 20; time 12.0, 0% solvent A, 100% solvent B, flow 20; time 14.0, 0% solvent A, 100% solvent B, flow 20; time 14.1, 100% solvent A, 0% solvent B, flow 20; time 19.1, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method C: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 90% solvent A, 10% solvent B, flow 20; time 10.0, 0% solvent A, 100% solvent B, flow 20; time 12.0, 0% solvent A, 100% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550. Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

The following intermediates may be prepared by the procedures described above:

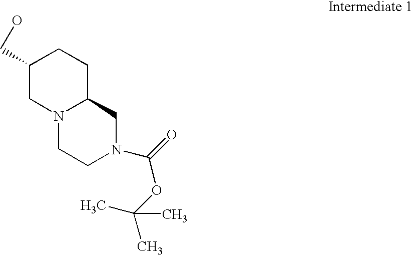

### trans-7-Hydroxymethyl-octahydro-pyrido[1,2-a]pyrazine-2-carboxylic acid tert-butyl ester

This compound was prepared in the manner described by M. Sanner et al (Bioorganic & Medicinal Chemistry Letters, 8:725-730 (1998)) and references cited therein.

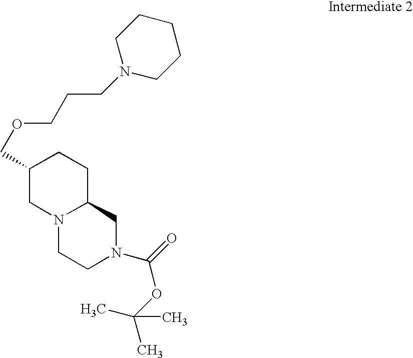

### trans-7-(3-Piperidin-1-ylpropoxymethyl)-octahydro-pyrido[1,2-a]pyrazine-2-carboxylic acid tert-butyl ester

A mixture of trans-7-hydroxymethyl-octahydro-pyrido[1,2-a]pyrazine-2-carboxylic acid tert-butyl ester (7.25 g, 26.85 mmol, intermediate 1 above) and 75 mL of THF was treated with 30 mL of 1.0 M solution potassium tert-butoxide (30 mmol, Aldrich Chemical Co.) and heated to 50° C. for 45 min. The solution was then treated with N-(3-chloropropyl)-piperidine (4.8 g, 28.8 mmol) in 25 mL of THF and the reaction mixture was heated to reflux overnight at which point a tlc showed conversion to the product. Removal of the solvent gave a viscous golden yellow syrup, 9.0 g.

Mass spectrum (m/z) calcd for C₂₂H₄₁N₃O₃: 395.48; obsd. 396.3 (M+1).

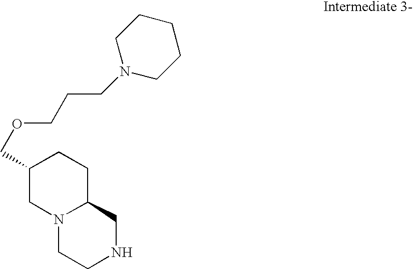

### trans-7-(3-Piperidin-1-ylpropoxymethyl)-octahydro-pyrido[1,2-a]pyrazine

The preceding ester of intermediate 2 (9.0 g) in 100 mL of methanol was treated with 20 mL of 4N HCl in dioxane (Aldrich Chemical Co.) and heated to a gentle reflux under a N₂ atmosphere overnight. After cooling to rt, the solvent was removed in vacuo to produce 5.63 g of a golden syrup.

Mass spectrum (m/z) calcd for C₁₇H33N₃O: 295.37; obsd 296.3 (M+1, 100%).

The same procedure can be used to prepare the cis isomer, cis-7-(3-piperidin-1-ylpropoxymethyl)-octahydro-pyrido[1,2-a]pyrazine.

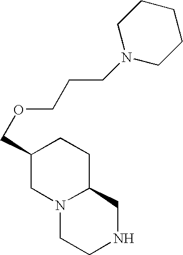

The following compounds may be prepared by the procedures below:

### EXAMPLE 1

### General Procedure A

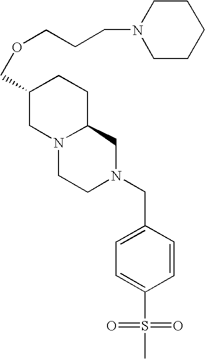

### trans-(7R, 9aS)-2-(4-Methanesulfonylbenzyl)-7-(3-piperidin-1-ylpropoxymethyl)-octa-hydro-pyrido[1,2-a]pyrazine

A solution of trans-7-(3-piperidin-1-ylpropoxymethyl)-octahydro-pyrido[1,2-a]pyrazine (5.63 g, 19.1 mmol) in 75 mL of acetic acid was treated with 4-methylsulfonylbenzaldehyde (10.53 g, 57.2 mmol, Aldrich Chemical Co.) at rt and stirred for an additional 1 hr. To this was added sodium triacetoxyborohydride (16.2 g, 76.4 mmol) portionwise over a 45 min period, after which the mixture was stirred at rt overnight. A tlc indicated good conversion to the desired product. The solvent was removed in vacuo and the residue was diluted with water, the pH adjusted to 9-10 with saturated aqueous Na₂CO₃, and extracted several times with methylene chloride. After drying with MgSO₄, the solvent was removed in vacuo to give the crude title product. This was flash chromatographed on silica gel using a gradient system of 2% CH₃OH in CH₂Cl₂ up to 1% saturated NH₄OH and 7% CH₃OH in CH₂Cl₂. Purified fractions were concentrated to a pale yellow syrupy residue. This free base was dissolved in a minimal volume of EtOAc and treated with HCl (1.0M in Et₂O, Aldrich Chemical Co.) to generate the hydrochloride salt as a white solid.

Mass spectrum (m/z) calcd for C₂₅H₄₁N₃O₃S: 463.38; obsd 464.3 (M+1, 100%).

¹H-nmr (CDCl₃, 400 MHz) δ 1.22 (m, 3H), 1.42 (m, 4H), 1.60 (m, 4H), 1.68-1.95 (m, 5H), 2.12 (m, 2H), 2.20 (m, 2H), 2.41 (bm, 4H), 2.57 (t, 2H), 2.70 (m, 2H), 3.03 (s, 3H), 3.41-3.60 (m, 6H), 7.52 (d, 2H), 7.85 (d, 2H).

Using the general procedure A, as described for Example 1 (with the exception of their conversion to a trifluoracetate salt), the following compounds were also prepared:

### EXAMPLE 2

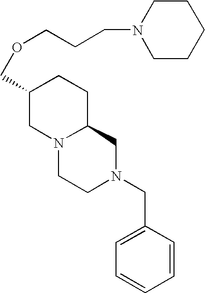

### trans-2-Benzyl-7-(3-piperidin-1-ylpropoxymethyl)-octahydro-pyrido[1,2-a]pyrazine

Mass spectrum (m/z) calcd for C₂₄H₃₉N₃O: 385.59; obsd: 386.3(M+1).

¹H-nmr (CDCl₃, 400 MHz) δ 0.96 (m, 1H), 1.23 (m, 1H), 1.47 (m, 4H), 1.64-1.83 (m, 12H), 1.93 (t, 2H), 2.26 (m, 2H), 2.44 (bs, 4H), 2.70 (t, 2h), 2.77 (d, 1H), 2.88 (d, 1H), 3.20 (m, 2H), 3.39 (m, 2H), 3.46 (d, 1 H), 7.28 (d, 5H).

### EXAMPLE 3

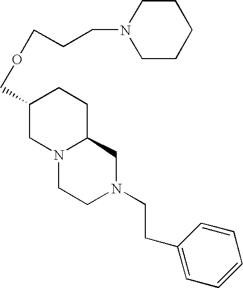

### trans-2-Phenethyl-7-(3-piperidin-1-ylpropoxymethyl)-octahydro-pyrido[1,2-a]pyrazine

Mass spectrum (m/z) calcd for C₂₅H₄₁N₃O: 399.62; obsd: 400.3 (M+1).

¹H-nmr (CDCl₃, 400 MHz) δ 0.96 (m, 2H), 1.24-1.28 (m, 2H), 1.44 (bs, 2H), 1.54-1.62 (m, 6H), 1.72-1.81 (m, 4H), 1.95 (m, 2H), 2.18 (m, 1 H), 2.29-2.37 (m, 1 H), 2.41 (bm, 6H), 2.59 (m, 1H), 2.75-2.79 (m, 2H), 2.85-2.95 (m, 2H), 3.20-3.24 (m, 2H), 3.38-3.43 (m, 2H), 7.18-7.27 (m, 5H).

### EXAMPLE 4

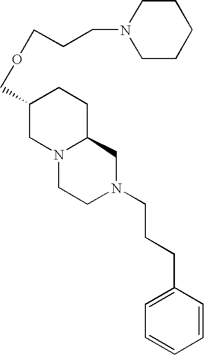

### trans-2-(3-Phenylpropyl)-7-(3-piperidin-1-ylpropoxymethyl)-octahydro-pyrido[1,2-a]pyrazine

Mass spectrum (m/z) calcd for C₂₆H₄₃N₃O: 413.65; obsd: 414.3 (M+1).

¹H-nmr (CDCl₃, 400 MHz) δ 0.96 (m, 1H), 1.22 (m, 1H), 1.43 (bs, 2H), 1.50 (d, 1H), 1.61 (bs, 2H), 1.77 (m, 10H), 1.91 (t, 2H), 2.20 (t, 2H), 2.31 (m, 2H), 2.42 (bs, 4H), 2.60 (t, 2H), 2.72 (d, 2H), 2.82 (d, 1H), 2.88 (d, 1H), 3.20 (m, 2H), 3.38 (q, 2H), 7.16-7.22 (m, 5H).

### EXAMPLE 5

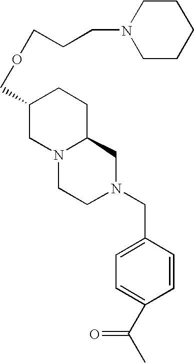

### trans-1-{4-[7-(3-Piperidin-1-ylpropoxymethyl)-octahydro-pyrido[1,2-a]pyrazin-2-ylmethyl]-phenyl}-ethanone

Mass spectrum (m/z) calcd for C₂₆H₄₁N₃O₂: 427.63; obsd: 428.3 (M+1).

¹H-nmr (CDCl₃, 400 MHz) δ 0.92-0.96 (m, 1H), 1.20-1.26 (m, 2H), 1.40 (bs, 2H), 1.46-1.76 (m, 13H), 1.82-1.95 (m, 3H), 2.25-2.34 (m, 6H), 2.57 (s, 3H), 2.63-2.65 (d, 1H), 2.70-2.75 (m, 1H), 2.88-2.92 (m, 1H), 3.16-3.22 (m, 2H), 3.34-3.41 (m, 2H), 3.50 (q, 2H), 7.39 (d, 2H), 7.89 (d, 2H).

### EXAMPLE 6

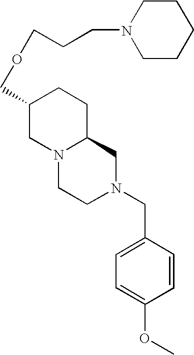

### trans-2-(4-Methoxybenzyl)-7-(3-piperidin-1-ylpropoxymethyl)-octahydro-pyrido[1,2-a]pyrazin-2-ylmethyl]-phenyl}-ethanone

Mass spectrum (m/z) calcd for C₂₅H₄₁N₃O₂: 415.62; obsd: 416.3 (M+1).

¹H-nmr (CDCl₃, 400 MHz) δ 0.95 (m, 1H), 1.20-1.24 (m, 1H), 1.43-1.49 (bs, 2H), 1.58-1.63 (m, 3H), 1.68-1.82 (m, 5H), 1.88-1.94 (m, 2H), 2.19-2.30 (m, 4H), 2.38-2,42 (m, 4H), 2.65-2.77 (d+m, 4H), 2.87-2.91 (d, 2H), 3.18-3.20 (m, 2H), 3.35-3.41 (m, 3H), 3.77 (s, 3H), 3.92 (m, 1H), 6.81-6.83 (d, 2H), 7.18-7.24 (d, 2H).

### EXAMPLE 7

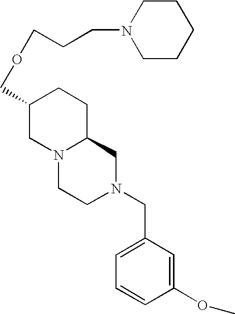

### trans-2-(3-Methoxybenzyl)-7-(3-piperidin-1-ylpropoxymethyl)-octahydro-pyrido[1,2-a]pyrazine

Mass spectrum (m/z) calcd for C₂₅H₄₁N₃O₂: 415.62; obsd: 416.3 (M+1).

### EXAMPLE 8

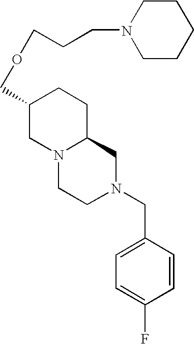

### trans-2-(4-Fluorobenzyl)-7-(3-piperidin-1-ylpropoxymethyl)-octahydro-pyrido[1,2-a]pyrazine

Mass spectrum (m/z) calcd for C₂₄H₃₈FN₃O: 403.58; obsd: 404.3 (M+1).

¹H-nmr (CDCl₃, 400 MHz) δ 0.96 (m, 1H), 1.20-1.24 (m, 1H), 1.42 (bs, 2H), 1.46-1.50 (m, 1H), 1.57-1.63 (m, 4H), 1.69-1.78 (m, 5H), 1.80-1.93 (m, 2H), 2.21-2.29 (m, 2H), 2.37-2.41 (m, 4H), 2.63-2.66 (dd, 2H), 2.69-2.75 (m, 2H), 2.88-2.91 (dd, 1H), 3.18-3.22 (m, 2H), 3.35-3.41 (m, 3H), 3.82 (t, 1H), 3.92 (m, 1H), 6.94-6.98 (m, 2H), 7.22-7.26 (m, 2H).

### EXAMPLE 9

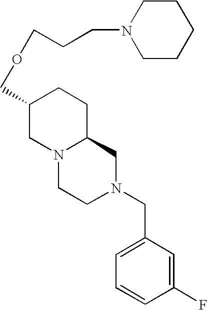

### trans-2-(3-Fluorobenzyl)-7-(3-piperidin-1-ylpropoxymethyl)-octahydro-pyrido[1,2-a]pyrazine

Mass spectrum (m/z) calcd for C₂₄H₃₈FN₃O: 403.58; obsd: 404.3 (M+1).

¹H-nmr (CDCl₃, 400 MHz) δ 0.94-1.03 (m, 1H), 1.22 (m, 1H), 1.49-1.51 (m, 3H), 1.69-1.98 (m, 8H), 2.10 (t, 1H), 2.23-2.34 (m, 2H), 2.62-2.77 (m, 6H), 2.88-2.92 (bs+m, 3H), 3.16-3.24 (m, 2H), 3.39-3.49 (m, 3H), 3.83 (t, 1H), 3.93-3.97 (m+bs, 2H), 6.88-6.93 (m, 1H), 7.02-7.05 (m, 2H), 7.21-7.26 (m,1H).

### EXAMPLE 10

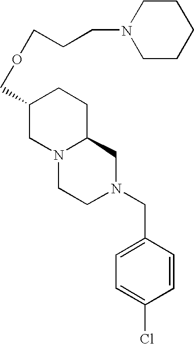

### trans-2-(4-Chlorobenzyl)-7-(3-piperidin-1-ylpropoxymethyl)-octahydro-pyrido[1,2-a]pyrazine

Mass spectrum (m/z) calcd for C₂₄H₃₈CIN₃O: 420.04; obsd: 420.3 (M+), 422.

¹H-nmr (CDCl₃, 400 MHz) δ 0.96 (m, 1H), 1.18-1.22 (m, 1H), 1.40 (bs, 2H), 1.46-1.50 (m, 1H), 1.53-1.59 (m, 3H), 1.69-1.83 (m, 6H), 1.87-1.92 (m, 2H), 2.10 (t, 1H), 2.21-2.35 (m, 6H), 2.62-2.74 (m, 2H), 2.88-2.91 (m, 1H), 3.18-3.22 (m, 2H), 3.35-3.42 (m, 3H), 3.82 (t, 1H), 3.92 (m, 2H), 7.21-7.26 (m, 4H).

### EXAMPLE 11

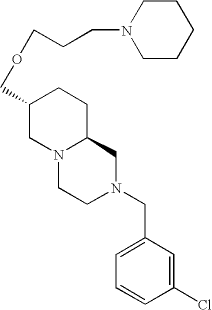

### trans-2-(3-Chlorobenzyl)-7-(3-piperidin-1-ylpropoxymethyl)-octahydro-pyrido[1,2-a]pyrazine

Mass spectrum (m/z) calcd for C₂₄H₃₈CIN₃O: 420.04; obsd: 420.3 (M+), 422.

¹H-nmr (CDCl₃, 400 MHz) δ 0.97 (m, 1H), 1.20-1.23 (m, 1H), 1.40 (bs, 2H), 1.47-1.60 (m, 4H), 1.70-1.85 (m, 5H), 1.92 (m, 2H), 2.15 (t, 1H), 2.22-2.37 (m, 5H), 2.63-2.75 (m, 3H), 2.88-2.92 (d+bs, 2H), 3.18-3.22 (m, 2H), 3.35-3.46 (m, 3H), 3.82 (t, 1H), 3.93 (m, 2H), 7.15-7.21 (m, ³H), 7.30 (s, 1H).

### EXAMPLE 12

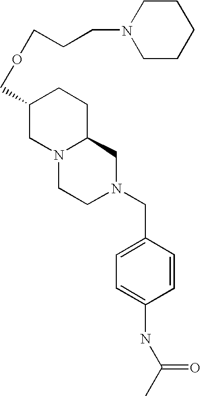

### trans-N-{4-[7-(3-Piperidin-1-ylpropoxymethyl)-octahydro-pyrido[1,2-a]pyrazin-2-ylmethyl]-phenyl}-acetamide

Mass spectrum (m/z) calcd for C₂₆H₄₂N₄O₂: 442.64; obsd: 443.3 (M+1).

¹H-nmr (CDCl₃, 400 MHz) δ 0.95 (m, 1H), 1.16-1.26 (m, 1H), 1.42 (bs, 2H), 1.47 (d, 1H), 1.54-1.59 (m, 3H), 1.69-1.82 (m, 6H), 1.90 (t, 2H), 2.14 (s, 3H), 2.23-2.36 (m, 5H), 2.64-2.75 (m, 4H), 2.88 (dd, 2H), 3.15-3.23 (m, 2H), 3.33-3.45 (m, 4H), 3.92-3.96 (m, 2H), 7.17 (s, 1H), 7.24 (m, 1), 7.40 (d, 2).

### EXAMPLE 13

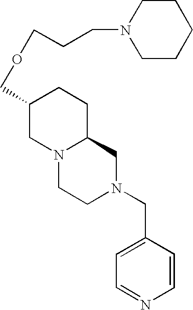

### trans-7-(3-Piperidin-1-yl-propoxymethyl)-2-pyridin-4-ylmethyl-octahydro-pyrido[1,2-a]pyrazine

Mass spectrum (m/z) calcd for C₂₃H38N₄O: 386.58; obsd: 387.3 (M+1).

¹H-nmr (CDCl₃, 400 MHz) δ 0.96 (m, 1H), 1.17-1.27 (m, 1H), 1.40 (bs, 2H), 1.46-1.58 (m, 5H), 1.68-1.77 (m, 7H), 1.83-1.96 (m, 3H), 2.23-2.34 (m, 6H), 2.63 (d, 1H), 2.70-2.74 (q, 2H), 2.89-2.92 (d, 2H), 3.16-3.24 (m, 2H), 3.33-3.49 (m+t, 4H), 7.24 (t, 2H), 8.51 (m, 2H).

### EXAMPLE 14

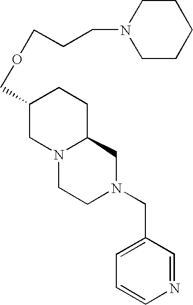

### trans-(3-Piperidin-1-ylpropoxymethyl)-2-pyridin-3-ylmethyl-octahydro-pyrido[1,2-a]pyrazine

Mass spectrum (m/z) calcd for C₂₃H₃₈N₄O: 386.58; obsd: 387.3 (M+1).

¹H-nmr (CDCl₃, 400 MHz) δ 0.96 (m, 1H), 1.23 (m, 1H), 1.40 (bs, 2H), 1.46-1.57 (m, 3H), 1.69-1.75 (m, 8H), 1.82-1.93 (m, 3H), 2.25-2.33 (m, 6H), 2.64 (d, 1H), 2.70-2.75 (m, 2H), 2.88-2.92 (dd, 1H), 3.18-3.21 (m, 2H), 3.34-3.41 (m, 2H), 3.47 (m, 2H), 7.21-7.23 (m, 1H), 7.62-7.64 (m, 1H), 8.46-8.51 (m, 2H).

### EXAMPLE 15

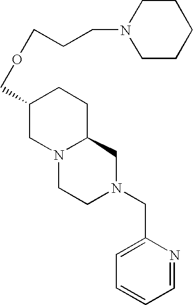

### trans-7-(3-Piperidin-1-yl-propoxymethyl)-2-pyridin-2-ylmethyl-octahydro-pyrido[1,2-a]pyrazine

Mass spectrum (m/z) calcd for C₂₃H₃₈N₄O: 386.58; obsd: 387.3 (M+1).

¹H-nmr (CDCl₃, 400 MHz) δ 0.93-1.01 (m, 1H), 1.20-1.29 (m, 1H), 1.42 (bs, 2H), 1.50-1.78 (m, 11H), 1.92-2.02 (m, 3H), 2.32-2.40 (m, 6H), 2.72-2.77 (m, 2H), 2.81 (d, 1H), 2.92 (m, 1H), 3.19-3.26 (m, 2H), 3.35-3.48 (m, 2H), 3.61-3.69 (t, 2H), 7.14-7.17 (m, 1H), 7.39 (d, 1H), 7.62-7.66 (m, 1H), 8.55-8.57 (m, 1H).

### EXAMPLE 16

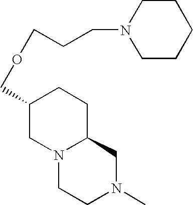

### trans-2-Methyl-7-(3-piperidin-1-ylpropoxymethyl)-octahydro-pyrido[1,2-a]pyrazine

Mass spectrum (m/z) calcd for C₁₈H₃₅N₃O: 309.50; obsd: 310.2 (M+1).

¹H-nmr (CDCl₃, 400 MHz) δ 0.98 (m, 1H), 1.24 (m, 2H), 1.42 (m, 2H), 1.51-1.82 (m, 9H), 1.90-1.93 (m, 2H), 2.18-2.27 (s+m, 4H), 2.41-2.45 (m, 5H), 2.64-2.75 (m, 3H), 2.88-2.91 (m, 1H), 3.17-3.211 (m, 2H), 3.38-3.41 (m, 2H).

### EXAMPLE 17

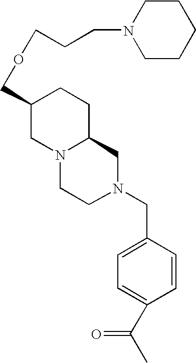

### cis-1-{4-[7-(3-Piperidin-1-ylpropoxymethyl)-octahydro-pyrido[1,2-a]pyrazin-2-ylmethyl]-phenyl}-ethanone

Mass spectrum (m/z) calcd for C₂₆H₄₁N₃O₂: 427.63; obsd: 428.4 (M+1), 296.

¹H-nmr (CDCl₃, 400 MHz) δ 0.96 (m, 1H), 1.22 (m, 2H), 1.40 (m, 2H), 1.48-1.57 (m, 3H). 1.67-1.76 (m, 6H), 1.83-1.94 (m, 3H), 2.22-2.33 (m, 7H), 2.57 (s, 3H), 2.63-2.75 (m, 3H), 2.90 (d, 1H), 3.16-3.22 (m, 2H), 3.35-3.41 (m, 2H), 3.45-3.55 (m, 2H), 7.39 (d, 2H), 7.89 (d, 2H).

### EXAMPLE 18

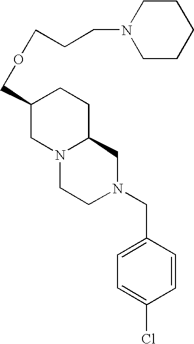

### cis-2-(4-Chlorobenzyl)-7-(3-piperidin-1-ylpropoxymethyl)-octahydro-pyrido[1,2-a]pyrazine

Mass spectrum (m/z) calcd for C₂₄H₃₈CIN₃O: 420.04; obsd: 420.3 (M+), 422.

¹H-nmr (CDCl₃, 400 MHz) δ 0.92-0.96 (m, 1H), 1.17-1.25 (m, 1H), 1.40-1.59 (m, 7H), 1.69-1.83 (m, 8H), 1.88-1.93 (m, 1H), 2.21-2.27 (m, 2H), 2.32-2.36 (m, 3H), 2.62-2.74 (m, 3H), 2.88-2.91 (m, 1H), 3.16-3.22 (m, 1H), 3.34-3.45 (m, 3H), 7.21-7.26 (m, 4H).

### EXAMPLE 19

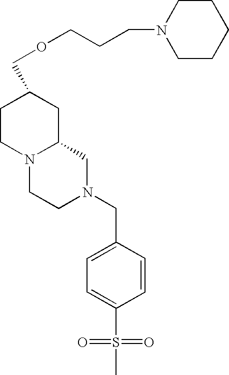

### cis-2-(4-Methanesulfonylbenzyl)-8-(3-piperidin-1-ylpropoxymethyl)-octahydro-pyrido[1,2-a]pyrazine

Mass spectrum (m/z) calcd for C₂₅H₄₁N₃O₃S: 463.68; obsd: 464.3 (M+1).

¹H-nmr (CDCl₃, 400 MHz) δ 0.90 (q, 1H), 1.27 (m, 1H), 1.45 (bs, 2H), 1.53 (d, 1H), 1.64-1.67 (m, 8H), 1.70-1.89 (m, 3H), 1.97-2.10 (bs+t, 2H), 2.25-2.33 (d, 2H), 2.46 (bm, 4H), 2.61 (d, 1H), 2.73 (d, 2H), 2.84 (d, 2H), 3.03 (s, 3H), 3.15-3.22 (m, 2H), 3.38-3.42 (m, 2H), 3.50-3.58 (t, 2H), 7.50 (d, 2H), 7.86 (d, 2H).

### EXAMPLE 20

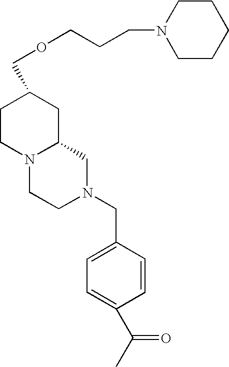

### cis-1-{4-[8-(3-Piperidin-1-ylpropoxymethyl)-octahydro-pyrido[1,2-a]pyrazin-2-ylmethyl]-phenyl}-ethanone

Mass spectrum (m/z) calcd for C₂₆H₄₁N₃O₂: 427.63; obsd: 428.3 (M+1).

¹H-nmr (CDCl₃, 400 MHz) δ 0.90 (m, 1H), 1.14-1.31 (m, 1H), 1.46 (bs, 2H), 1.52 (d, 1H), 1.63-1.70 (m, 8H), 1.83-1.88 (t, 3H), 1.99-2.10 (m, 2H), 2.27-2.35 (m, 2H), 2.52 (bm, 4H), 2.58-2.65 (m, 1H), 2.74 (m, 2H), 2.84-2.87 (d, 1H), 3.15-3.22 (m, 2H), 3.38-3.42 (m, 2H), 3.47-3.55 (m, 2H), 7.39 (d, 2H), 7.88 (d, 2H).

### EXAMPLE 21

### General Procedure B

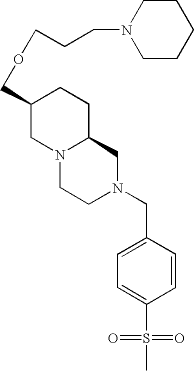

### cis-2-(4-Methanesulfonylbenzyl)-7-(3-piperidin-1-ylpropoxymethyl)-octahydro-pyrido[1,2-a]pyrazine

Under N₂, a mixture of powdered KOH (31 mg, 0.55 mmol) and tetrabutylammonium bromide (32 mg, 0.1 mmol) in 3 mL of THF was treated with a solution of cis-7-(3-piperidin-1-ylpropoxymethyl)-octahydro-pyrido[1,2-a]pyrazine (148 mg, 0.3 mmol, prepared in a similar manner to the trans isomer as described in intermediate 3) in 3 mL of THF. After 10 min, 4-(methylsulfonyl)benzyl chloride (103 mg, 0.5 mmol) was added with stirring at rt for an additional 6 hr, at which time a tlc showed good conversion to the product. The mixture was concentrated in vacuo to dryness and the residue was redissolved in CH₂Cl₂, washed with saturated aqueous NaHCO₃, saturated NaCl and dried over Na₂SO₄. The solvent was removed in vacuo to a viscous yellow oil. Flash chromatography using a gradient of 5% MeOH in CH₂Cl₂ to 0.5% NH₄OH and 5% MeOH in CH₂Cl₂ gave a clear viscous oil, 67 mg. This was converted to the hydrochloride salt using 1.0M HCl in Et₂O as described above to produce a white solid, 64 mg.

Mass spectrum (m/z) calcd for C₂₅H₄₁N₃O₃S: 463.38; obsd 464.3 (M+1, 100%), 296.

¹H-nmr (CDCl₃, 400 MHz) δ 1.40-1.76 (m, 16H), 1.83-1.92 (m, 2H), 2.27 (d, 1), 2.35 (bm, 4H), 2.62 (m, 1H), 2.71 (m, 2H), 2.90 (m, 1H), 3.03 (s, 3H), 3.20 (m, 2H), 3.39 (m, 2H), 3.53 (m, 2H), 7.52 (d, 2H), 7.85 (d, 2H).

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris HCl containing 2 mM MgCl₂ (pH to 7.4 at 4 degrees C.). The homogenate is then centrifuged at 45,000 G for 10 minutes. The supernatant is decanted and the membrane pellet re-suspended by Polytron in cold 50 mM Tris HCl containing 2 mM MgCl₂ (pH to 7.4 at 4 degrees C.) and centrifuged again. The final pellet is re-suspended in 50 mM Tris HCl containing 2 mM MgCl₂ (pH to 7.4 at 25 degrees C.) at a concentration of 12 mg/mL. Dilutions of compounds are made in 10% DMSO/50 mM Tris buffer (pH 7.4) (at 10×final concentration, so that the final DMSO concentration is 1%). Incubations are initiated by the addition of membranes (200 microliters) to 96 well V-bottom polypropylene plates containing 25 microliters of drug dilutions and 25 microliters of radioligand (1 nM final concentration ³H-N-methylhistamine). After a 1 hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be determined for each dose of the compound, and an IC50 or Ki value can be calculated from these results.

TABLE 1
Rat H3 Binding for selected compounds

| Example # | Rat H3 activity (Kᵢ, nM) |
|---|---|
| 1 | 42 |
| 2 | 125 |
| 3 | 54 |
| 4 | 37 |
| 5 | 35 |
| 6 | 55 |
| 7 | 28 |
| 8 | 26 |
| 9 | 9 |
| 10 | 14 |
| 11 | 33 |
| 12 | 70 |
| 13 | 33 |
| 14 | 69 |
| 15 | 90 |
| 16 | 114 |
| 17 | 60 |
| 18 | >69 |
| 21 | 26 |
| Intermediate 3 | 155 |

1. A compound of formula I or II 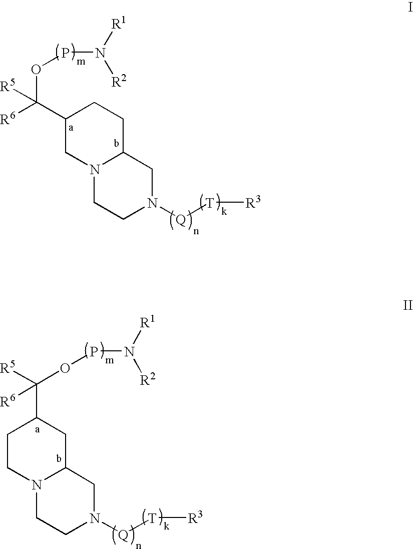
or the pharmaceutically acceptable salt(s) thereof, wherein:
P is independently methylene (optionally substituted at available positions on the carbon atom by hydrogen, fluorine, OH, carbonyl, C₁-C₈ alkylene); or a 3-8 membered cycloalkyl ring (e.g., cyclopropyl, cyclobutyl, cyclopentyl, cyclohexyl);
m=2, 3 or 4;
Q is independently selected from C₁-C₆ alkylene and may be substituted at available positions by R^{a} wherein R^{a} is selected from hydrogen, C₁-C₆ alkyl or C₆-C₁₄ aryl;
T is selected from the group consisting of methylene, C═O, —C(═O)—N, SO₂;
n=0, 1, 2, or 3;
k=0, 1;
R¹ and R² are independently selected from the group consisting of hydrogen;
C₁-C₈ alkyl optionally substituted with 1 to 4 halogens (especially fluorine) or OH;
C₃-C₇ cycloalkyl;
C₆-C₁₄ aryl;
3-8 membered heterocycloalkyl optionally substituted with a C₁-C₄ alkyl-carbonyl group;
C₆-C₁₀ arylsulfonyl optionally substituted with C₁-C₂ alkyl; and
5-10 membered heteroaryl; or
NR¹ together with an available carbon atom in P form a 4-8 membered cyclic ring, said ring may include up to two additional heteroatoms selected from N, O or S;
R³ is selected from the group consisting of hydrogen;
phenyl, naphthyl or heteroaryl, each of which may be optionally substituted by one or more of X; wherein
X is H, F, Cl, Br, I, CN, OH, OR⁷, S(O)ₜR⁸, COR⁹, CONR¹⁰R¹¹; or
R¹ and R² together with the nitrogen of the NR¹R² group form a 4-8 member ring, wherein from one to three of the carbons in the ring is optionally replaced by O, S, NR⁴, or CO, and the ring is optionally fused to a C₆-C₁₀ arylene and is optionally substituted at a ring carbon with one or two C₁-C₄ alkyl groups;
R⁴is hydrogen;
C₁-C₈ alkyl optionally substituted with 1 to 4 halogens;
5-10 membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, and cyano;
C₆-C₁₀ aryl optionally substituted with one or two C₁-C₂ alkyl; or
C₁-C₄ alkyl-carbonyl;
R⁵ and R⁶ are independently selected from the group consisting of hydrogen;
fluorine;
C₁-C₆ alkyl (optionally substituted by F); or
R⁵ and R⁶ together with the carbon atom to which they are attached form a cycloalkyl ring of 3 to 7 atoms;
R⁷, R⁸ and R⁹ are independently selected from the group consisting of C₁-C₈ alkyl optionally substituted with 1 to 4 halogens;
5-10 membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, and cyano;
C₆-C₁₀ aryl optionally substituted with one or two C₁-C₂ alkyl;
R¹⁰ and R¹¹ are independently selected from the group defined as R¹ and R² above; and
t is 0, 1 or 2.

2. The compound of claim 1, wherein R¹ is methyl, R² is methyl, and R³ is phenyl.

3. The compound of claim 1, wherein R¹ and R² together with the nitrogen to which they are attached form the 5-membered pyrrolidine ring and R³ is phenyl.

4. The compound of claim 1, wherein R¹ and R² together with the nitrogen to which they are attached form a 5-membered pyrrolidine ring and the groups at carbon positions a and b are trans.

5. The compounds of claim 1 together with the nitrogen to which they are attached from the 6-membered piperidine ring and R³ is phenyl and the groups at position a and b are trans.

6. A compound of the formula

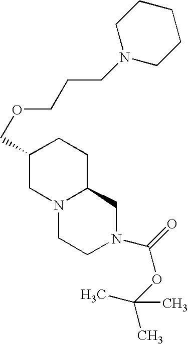

7. A trans compound of the formula

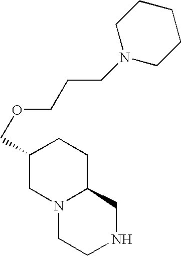

8. A cis compound of the formula

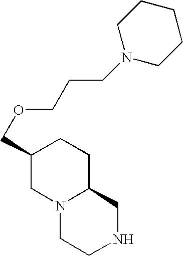

9. The compounds of formula I or II in accordance with the present invention are the following:
(7R, 9a S)-7-(3-Piperidin-1-ylpropoxymethyl)-octahydro-pyrido[1,2-a]pyrazine;
(7R, 9aS)-2-Methyl-7-(3-piperidin-1-ylpropoxymethyl)-octahydro-pyrido[1,2-a]pyrazine;
(7R, 9aS)-2-(3-Phenylpropyl)-7-(3-piperidin-1-ylpropoxymethyl)-octahydro-pyrido[1,2a]pyrazine;
(7R, 9aS)-2-Phenethyl-7-(3-piperidin-1-ylpropoxymethyl)-octahydro-pyrido[1,2-a]pyrazine;
(7R, 9aS)-2-Benzyl-7-(3-piperidin-1-ylpropoxymethyl)-octahydro-pyrido[1,2-a]pyrazine;
(7R, 9aS)-1-{4-[7-(3-Piperidin-1-ylpropoxymethyl)-octahydro-pyrido-[1,2-a]pyrazin-2-ylmethyl]-phenyl}-ethanone;
(7R, 9aS)-2-(4-Methoxybenzyl)-7-(3-piperidin-1-ylpropoxymethyl)-octahydro-pyrido[1,2-a]pyrazine;
(7R, 9aS)-2-(3-Methoxybenzyl)-7-(3-piperidin-1-ylpropoxymethyl)-octahydro-pyrido[1,2-a]pyrazine;
(7R, 9aS)-2-(4-Fluorobenzyl)-7-(3-piperidin-1-ylpropoxymethyl)-octahydro-pyrido[1,2-a]pyrazine;
(7R, 9aS)-N-{4-[7-(3-Piperidin-1-ylpropoxymethyl)-octahydro-pyrido-[1,2-a]pyrazin-2-ylmethyl]-phenyl}-acetamide;
(7R, 9aS)-2-(4-Chlorobenzyl)-7-(3-piperidin-1-ylpropoxymethyl)-octahydro-pyrido[1,2-a]pyrazine;
(7R, 9aS)-2-(3-Chlorobenzyl)-7-(3-piperidin-1-ylpropoxymethyl)-octahydro-pyrido[1,2-a]pyrazine;
(7R, 9aS)-2-(3-Fluorobenzyl)-7-(3-piperidin-1-ylpropoxymethyl)-octahydro-pyrido[1,2-a]pyrazine;
(7R, 9aS)-2-(4-Methanesulfonylbenzyl)-7-(3-piperidin-1-ylpropoxy-methyl)-octahydro-pyrido[1,2-a]pyrazine;
(7S, 9aS)-1-{4-[7-(3-Piperidin-1-ylpropoxymethyl)-octahydro-pyrido-[1,2-a]pyrazin-2-ylmethyl]-phenyl}-ethanone;
(7S, 9aS)-2-(4-Chlorobenzyl)-7-(3-piperidin-1-ylpropoxymethyl)-octahydro-pyrido[1,2-a]pyrazine;
(7R, 9aS)-7-(3-Piperidin-1-ylpropoxymethyl)-2-pyridin-3-ylmethyl-octahydro-pyrido[1,2-a]pyrazine;
(7R, 9aS)-7-(3-Piperidin-1-ylpropoxymethyl)-2-pyridin-4-ylmethyl-octahydro-pyrido[1,2-a]pyrazine;
(7R, 9aS)-7-(3-Piperidin-1-ylpropoxymethyl)-2-pyridin-2-ylmethyl-octahydro-pyrido[1,2-a]pyrazine;
(7S, 9aS)-2-(4-Methanesulfonylbenzyl)-7-(3-piperidin-1-ylpropoxymethyl)-octahydro-pyrido[1,2-a]pyrazine;
(8R, 9aS)-2-(4-Methanesulfonylbenzyl)-8-(3-piperidin-1-ylpropoxymethyl)-octahydro-pyrido[1,2-a]pyrazine; and
(8R, 9aS)-1-{4-[8-(3-Piperidin-1-ylpropoxymethyl)-octahydro-pyrido-[1,2-a]pyrazin-2-ylmethyl]-phenyl}-ethanone.

10. The compound of formula I or II, wherein the compound is selected from the group consisting of:
2-(4-Methanesulfonylbenzyl)-7-(3-morpholin-4-ylpropoxymethyl)-octahydro-pyrido[1,2-a]pyrazine;
7-[3-(2,5-Dimethylpyrrolidin-1-yl)-propoxymethyl]-2-(4-methanesulfonylbenzyl)-octahydro-pyrido[1,2-a]pyrazine;
2-(4-Methanesulfonylbenzyl)-7-[3-(4-methylpiperazin-1-yl)-propoxymethyl]-octahydro-pyrido[1,2-a]pyrazine;
2-(4-Methanesulfonylbenzyl)-7-[3-(4-phenylpiperazin-1-yl)-propoxymethyl]-octahydro-pyrido[1,2-a]pyrazine;
{3-[2-(4-Methanesulfonylbenzyl)-octahydro-pyrido[1,2-a]pyrazin-7-ylmethoxy]-propyl}-dimethylamine;
7-(3-Azetidin-1-yl-propoxymethyl)-2-(4-methanesulfonylbenzyl)-octahydro-pyrido[1,2-a]pyrazine;
2-(4-Methanesulfonylbenzyl)-7-(3-thiomorpholin-4-ylpropoxymethyl)-octahydro-pyrido[1,2-a]pyrazine;
7-[3-(3,4-Dihydro-1H-isoquinolin-2-yl)-propoxymethyl]-2-(4-methanesulfonylbenzyl)-1,2-a]pyrazine;
7-[3-(3,4-Dihydro-2H-quinolin-1-yl)-propoxymethyl]-2-(4-methanesulfonylbenzyl)-octahydro-pyrido[1,2-a]pyrazine;
7-[3-(2,6-Dimethyl-piperidin-1-yl)-propoxymethyl]-2-(4-methanesulfonylbenzyl)-octahydro-pyrido[1,2-a]pyrazine;
2-(4-Methanesulfonylbenzyl)-7-(2-piperidin-1-ylethoxymethyl)-octahydro-pyrido[1,2-a]pyrazine;
2-(4-Methanesulfonylbenzyl)-7-(2-morpholin4-ylethoxymethyl)-octahydro-pyrido[1,2-a]pyrazine;
(4-Methanesulfonylphenyl)-[7-(3-piperidin-1-ylpropoxymethyl)-octahydro-pyrido[1,2-a]pyrazin-2-yl]-methanone;
2-(4-Methanesulfonylbenzenesulfonyl)-7-(3-piperidin-i -ylpropoxymethyl)-octahydro-pyrido[1,2-a]pyrazine;
7-(3-Piperidin-1-ylpropoxymethyl)-octahydro-pyrido[1,2-a]pyrazine-2-carboxylic acid (4-methanesulfonylphenyl)-amide;
Phenyl-[8-(3-piperidin-1-ylpropoxymethyl)-octahydro-pyrido[1,2-a]pyrazin-2-yl]-methanone;
2-Benzenesulfonyl-8-(3-morpholin-4-ylpropoxymethyl)-octahydro-pyrido[1,2-a]pyrazine;
8-(3-Morpholin-4-ylpropoxymethyl)-2-(pyridine-4-sulfonyl)-octahydro-pyrido[1,2-a]pyrazine;
(2-Methylpyrimidin-5-yl)-[8-(3-morpholin-4-ylpropoxymethyl)-octahydro-pyrido[1,2-a]pyrazin-2-yl]-methanone;
1-(4-{1-[8-(3-Morpholin-4-ylpropoxymethyl)-octahydro-pyrido[1,2-a]pyrazin-2-yl]-ethyl}-phenyl)-ethanone;
Cyclopropyl-(4-{7-[1-(3-piperidin-1-ylpropoxy)-ethyl]-octahydro-pyrido[1,2-a]pyrazin-2-ylmethyl}-phenyl)-methanone;
7-(3-Piperidin-1-ylpropoxymethyl)-2-quinolin-8-ylmethyl-octahydro-pyrido[1,2-a]pyrazine;
5-[7-(3-Piperidin-1-ylpropoxymethyl)-octahydro-pyrido[1,2-a]pyrazin-2-ylmethyl]-indan-1-one; and
7-[7-(3-Piperidin-1-ylpropoxymethyl)-octahydro-pyrido[1,2-a]pyrazin-2-ylmethyl]-chroman-4-one.

11. A pharmaceutical composition for treating a disorder or condition that may be treated by antagonizing histamine-3 receptors, the composition comprising a compound of formula I or II as described in claim 1, and optionally a pharmaceutically acceptable carrier.

12. A method of treatment of a disorder or condition that may be treated by antagonizing histamine-3 receptors, the method comprising administering to a mammal in need of such treatment a compound of formula I or II as described in claim 1.

13. A pharmaceutical composition comprising a compound of formula I or II as described in claim 1, and optionally a pharmaceutically acceptable carrier.

14. A method of treatment of a disorder or condition selected from the group consisting of depression, mood disorders, schizophrenia, anxiety disorders, Alzheimer's disease, attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I or II as described in claim 1.

15. The method of claim 14, wherein the disorder or condition is selected from the group consisting of anxiety disorders, attention-deficit hyperactivity disorder, respiratory diseases, and obesity.

16. The method of claim 14, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

17. A pharmaceutical composition for treating allergic rhinitis, nasal congestion or allergic congestion comprising:
a) an H3 receptor antagonist compound of formula I or II; or a pharmaceutically acceptable salt thereof;
b) an H1 receptor antagonist such as cetirizine; or a pharmaceutically acceptable salt thereof; and
c) a pharmaceutically acceptable carrier;
wherein the active ingredients (a) and (b) above are present in amounts that render the composition effective in treating allergy rhinitis, nasal congestion or allergic congestion.

18. A pharmaceutical composition for treating depression and mood disorder comprising:
a) an H3 receptor antagonist or a pharmaceutically acceptable salt thereof;
b) a neurotransmitter uptake blocker or a pharmaceutically acceptable salt thereof;
c) and a pharmaceutically acceptable carrier;
wherein the active ingredients (a) and (b) above are present in amounts that render the composition effective in treating depression and mood disorder.

19. The composition according to claim 18 wherein the H3 receptor antagonist and the neurotransmitter blocker are given simultaneously.

20. The composition according to claim 17 wherein the H3 receptor antagonist and the H1 receptor antagonist are given simultaneously.

21. The pharmaceutical composition of claim 18 wherein the neurotransmitter uptake blocker is selected from the group consisting of sertraline, fluoxetine and paroxetine.
